(19) United States
(12) Patent Application Publication
(10) Pub. No.: US 2016/0299133 A1
(43) Pub. Date: Oct. 13, 2016

(54) FULL-AUTOMATIC INSTANT CHECK METER

(71) Applicant: HANGZHOU JOINSTAR BIOMEDICAL TECHNOLOGY CO., LTD., Hangzhou, Zhejiang (CN)

(72) Inventors: Xuyi ZHOU, Hangzhou, Zhejiang Province (CN); Longbin HONG, Hangzhou, Zhejiang Province (CN); Wenjie XU, Hangzhou, Zhejiang Province (CN); Xiaodong CHEN, Hangzhou, Zhejiang Province (CN); Songbing ZHOU, Hangzhou, Zhejiang Province (CN); Guojin SHEN, Hangzhou, Zhejiang Province (CN); Rui HU, Hangzhou, Zhejiang Province (CN)

(21) Appl. No.: 15/101,127

(22) PCT Filed: Dec. 25, 2014
(86) PCT No.: PCT/CN2014/094931

§ 371 Date: Jun. 2, 2016

(30) Foreign Application Priority Data
Dec. 26, 2013 (CN) 201310730567.X

(51) Int. Cl. G01N 33/543; G01N 35/04; G01N 35/00

(52) U.S. Cl. CPC G01N 33/54373; G01N 35/0099; G01N 35/04; G01N 2035/0412; G01N 2035/0427; G01N 2035/00148

(57) ABSTRACT

A full-automatic instant check meter, comprising a shell, a frame body (1), a sample rack (2), a sample input device, a shaking device, a detection card stack, a sample reaction device, a detection card conveying device, a detection device and a control panel, wherein the sample input device, the shaking device, the detection card stack, the sample reaction device, the detection card conveying device and the detection device are all arranged on the frame body (1); the sample rack (2) is arranged in the sample input device, and a sample test tube (3) is placed on the sample rack (2); the shaking device is arranged behind the sample input device; and the sample reaction device, the detection card conveying device and the detection device are all arranged behind the shaking device. The full-automatic instant check meter has a simple structure and high integration and automation degrees, and supports non-stop loading.

### FIELD OF THE INVENTION

The present invention relates to a technical field of medical testing apparatus, in particular, to a full-automatic claim instant check meter.

### BACKGROUND OF THE INVENTION

POCT product is a branch of in vitro diagnostics (IVD) industry; with its convenient and fast advantages, the product is clinically popular to quickly obtain the results of point of care testing. Currently the POCT Meters have been widely used in hospitals, clinics and patients' homes, which can detect most routine clinical indicators. At present, the global market scale of POCT meters has exceeded 40 billion US dollars. From 1980s to 1990s, during the early development period of the industry, the POCT meters have achieved an annual growth rate of nearly 30% in the Europe and America. With the economical development and increasing population aging trend in china, the incidences of rheumatoid arthritis, diabetes, cardiovascular and cerebrovascular diseases increase year by year and POCT meters will be increasingly widely used in clinics. With the implementation of China's new medical reform, the popularization of medical insurance and increased knowledge of disease prevention and health, the demands of POCT meters will be greater in health care, disease prevention and monitoring, playing an important role.

The advances in medical science greatly depend on relevant laboratory tests. Currently there are many automation equipments available on the markets, with high degree of automation and high detection capacity; but they require complicated sample pre-treatment processes and a long time of detection cycle (Turn around Time, TAT). The existing POCT meters can achieve a miniature, immediate and simple bedside testing, but POCT meter has a common feature—single test; if there are many clinical specimens in batches or the number of specimens for the same test item in the emergency testing is large, the single, small POCT testing instruments can not meet their requirements; moreover, the individual difference for signal operation is great, and the result has high dispersion degree, the quality is difficult to control, which is not conducive to the consistency of the test results in the same medical institute; in addition, it requires manual operation and takes a long time, increase the mean TAT for a single test; and the single tests are scattered, with high average cost.

### SUMMARY OF THE INVENTION

The invention provides a full-automatic instant check meter, which has a simple structure and high integration and automation degrees, and supports non-stop loading.

The invention is achieved through the following technical solutions:

A fully-automatic instant check meter, comprising a shell, a frame body, a sample rack, a sample input device, a shaking device, a detection card stack, a sample reaction device, a detection card conveying device, a detection device and a control panel, wherein the sample input device, the shaking device, the detection card stack, the sample reaction device, the detection card conveying device and the detection device are all arranged on the frame body; the sample rack is arranged in the sample input device, and a sample test tube is placed on the sample rack; the shaking device is arranged behind the sample input device; and the sample reaction device, the detection card conveying device and the detection device are all arranged behind the shaking device. The sample input device comprises a push rod mechanism, a limiting mechanism and an induction mechanism; the shaking device comprises a first barcode printer, a rotating mechanism, a vacuum blood vessel shaking mechanism and a peripheral blood vessel shaking mechanism; and the rotating mechanism, vacuum blood vessel shaking mechanism and peripheral blood vessel shaking mechanism are all fixed on the frame body; the peripheral blood vessel shaking mechanism is arranged below the vacuum blood vessel shaking mechanism; the rotating mechanism is arranged on the side of vacuum blood vessel shaking mechanism; the detection card stack comprises a detection card, a detection card stack and a scanning mechanism; the detection card is arranged in the detection card stack, and the scanning mechanism is arranged behind the detection card stack; the sample reaction device comprises a reaction cup, a flowing water ring, a reagent bin, a reagent bottle, a sampling arm mechanism, a gripping mechanism, a reaction cup stack area and a metal bath area; the reagent bottle is arranged in the reagent bin; the flowing water ring is arranged on the upper end face of the reagent bin, and the reaction cup is arranged above the flowing water ring; the sampling arm mechanism is arranged above the reagent bin and fixed on the frame body. The gripping mechanism is arranged above the reaction cup stack area and metal bath area and fixed on the frame body; the detection card conveying device comprises a detection card conveying arm, a buffer cartridge and a card extraction mechanism; the detection card conveying arm is arranged behind the detection card stack and fixed on the frame body; the card extraction mechanism is arranged on the detection card conveying arm in a sliding manner; the detection device comprises a detection mechanism and a detection card throwing mechanism; the detection mechanism is arranged above the detection card throwing mechanism; the buffer cartridge is arranged between the detection mechanism and the card extraction mechanism; the control panel is fixed to the frame body; the push rod mechanism, limiting mechanism, induction mechanism, first barcode printer, rotating mechanism, vacuum blood vessel shaking mechanism, peripheral blood vessel shaking mechanism, scanning mechanism, sampling arm mechanism, gripping mechanism, metal bath area, detection card conveying arm, buffer cartridge, card extraction mechanism, detection mechanism and detection card throwing mechanism are electrically connected on the control panel respectively.

The push rod mechanism comprises a first push rod, a second push rod, a third push rod and a fourth push rod, respectively driven by the first motor, second motor, third motor and fourth motor; the first push rod and the third push rod are used to move the sample rack longitudinally, and the second push rod and the fourth push rod are used to move the sample rack transversely; the limiting mechanism comprises the first limit switch that limits the sample rack longitudinally and the second limit switch that limits the sample rack transversely; the sample input device further comprises a limiting door for butting and a beam photoelectric sensor is arranged above the limiting door.

The rotating mechanism comprises a first DC motor and a rotating friction head; the first DC motor is connected with the frame body in sliding manner at the vertical direction; the vacuum blood vessel shaking mechanism comprises a guide block and a gripper, the guide block is fixed on the frame body and the gripper is connected with the frame body in sliding manner at the vertical direction; a check block is provided on the gripper and the check block rotates to drive the gripper to rotate under the guiding of the guide block; the peripheral blood vessel shaking mechanism comprises an installation platform, a second DC motor, a lead screw, a fixed block, a brushless motor and a fixing piece; the installation platform is fixed on the frame body, the fixed block is connected with the installation platform in a sliding manner driven by the second DC motor and the lead screw; an eccentric block is arranged on the brushless motor to drive the fixing piece to vibrate when the brushless motor rotates.

The reagent bin is arranged on the frame body; the reagent bin is internally provided with a fifth motor and a first gear is arranged at the output end of the fifth motor. A reagent tray is arranged in the reagent bin and a ring gear is arranged on the lower surface of the reagent tray. The first gear is meshed with the ring gear; the reagent bottle is arranged on the reagent tray; the flowing water ring is evenly distributed with four reaction cup jacks; the reaction cup is inserted into the reaction cup jacks; the flowing water ring is provided with card latch and a sixth motor outside. The output upper end of the sixth motor is provided with a second gear which is meshed with the flowing water ring; the metal bath area is located at the side of the reagent bin, the reaction cup stack area is located at the side of the metal bath area; an opening is arranged on the reagent bin; the second barcode printer is arranged at the opening and fixed on the frame body; a radiator is arranged on the side of the fifth motor inside the reagent bin; chilling chips are arranged above the radiator and fans are arranged below the radiator.

The gripping mechanism comprises a gripper arm, an electromagnet, a sliding block, a left grab block and a right grab block; a seventh motor and a sliding rail are arranged on the gripper arm, the sliding block is connected with the sliding rail in a sliding manner and connected the seventh motor via a synchronous belt; the electromagnet is arranged on the sliding block and a first connecting rod is arranged at the lower end of the electromagnet; the left grab block and right grab block are pivotally connected with the first connecting rod via the second connecting rod respectively.

A reagent bin cover is arranged above the reagent bottle and the reagent bin cover is provided with a sector-shaped notch.

The detection module comprises one or more of an immune colloidal gold detection module, an immunofluorescence detection module, an IMB detection module, and an immunofluorescence particle module; the immune colloidal gold detection module or immunofluorescence detection module or IMB detection module or immunofluorescence particle module is arranged above the detection card bracket.

The invention can achieve the following beneficial effects:

In the present invention, the full-automatic instant check meter integrates the immune colloidal gold technology and immunofluorescence technology on a single instrument. It provides a full-automatic, multi-item rapid immunoassay analyzer, having the advantage of high integration and automation degrees, and supporting the non-stop loading, etc. It can achieve one-time completion of sampling, testing, data processing and result report, which not only overcomes the shortcomings of complicated manual operation and slow speed, but also overcomes the disadvantages of a long TAT and specimen pre-treatment for large-scale automated instrument testing. POCT can achieve the quality control more feasible after automatic batch testing; moreover, our developed instruments and reagents are far cheaper than the imported products according to the characteristics of many patients in Chinese hospitals, heavy workloads and low costs, which are more suitable for China's national conditions. The full-automatic instant check meter overcomes the shortcomings of complicated specimen pre-treatment and many processes of the large-scale automated instruments; besides, it can achieve automatic continuous testing, greatly shortens the average test cycle of each test, and resolves the shortcomings of time-consuming single manual operation and unable to batch testing. The full-automatic instant check meter can meet the testing of single item or multiple items, single specimen or batch specimens in hospital outpatient clinics.

### BRIEF DESCRIPTION OF THE DRAWINGS

The invention is further described in combination with the drawings.

FIG. 1 is a schematic view of a full-automatic instant check meter under the shell removed state.

FIG. 2 is a schematic view of a sample input device in the present invention.

FIG. 3 is a schematic view of a shaking device in the present invention.

FIG. 4 is a schematic view of a sample reaction device in the present invention.

FIG. 5 is an exploded view of a reagent bin in the present invention.

FIG. 6 is a schematic view of a gripping mechanism in the present invention.

The names of parts in the figures are as follows:

1, frame body; 2, sample rack; 3, sample test tube; 4, first barcode printer; 5, detection card; 6, detection card stack; 7, reaction cup; 8, flowing water ring; 9, reagent bin; 10, reagent bottle; 11, reaction cup stack area; 12, metal bath area; 13, buffer cartridge; 14, first push rod; 141, first motor; 15, second push rod; 151, second motor; 16, third push rod; 161, third motor; 17, fourth push rod; 171, fourth motor; 18, first limit switch; 19, second limit switch; 20, limiting door; 21, beam photoelectric sensor; 22, first DC motor; 23, rotating friction head; 24, guide block; 25, gripper; 26, check block; 27, installation platform; 28, second DC motor; 29, lead screw; 30, fixed block; 31, brushless motor; 32, fixing piece; 33, eccentric block; 34, fifth motor; 35, first gear; 36, reagent tray; 37, ring gear; 38, reaction cup jack; 39, card latch; 40, sixth motor; 41, second gear; 42, opening; 43, second barcode printer; 44, radiator; 45, chilling chip; 46, fan; 47, gripper arm; 48, electromagnet; 49, sliding block; 50, left grab block; 51, right grab block; 52, seventh motor; 53, sliding rail; 54, synchronous belt; 55, first connecting rod; 56, second connecting rod; 57, reagent bin cover; 58, sector-shaped notch.

### DETAILED DESCRIPTION OF THE EMBODIMENTS

The invention is further described in details in combination with drawings and embodiments.

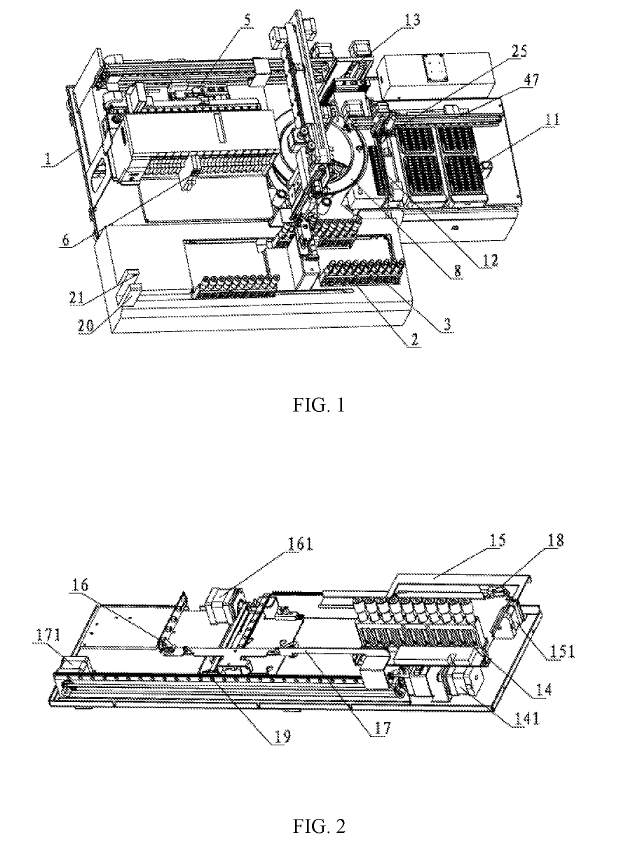

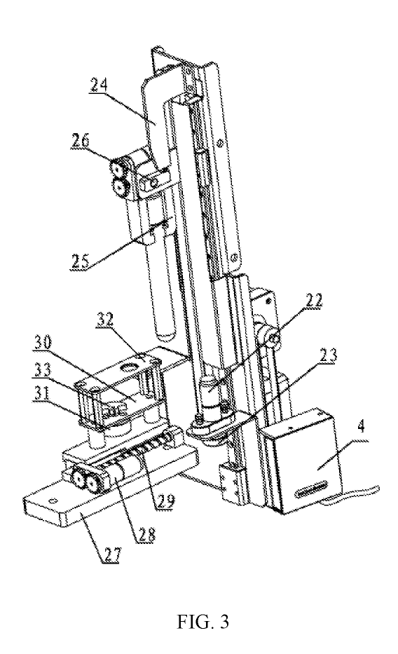

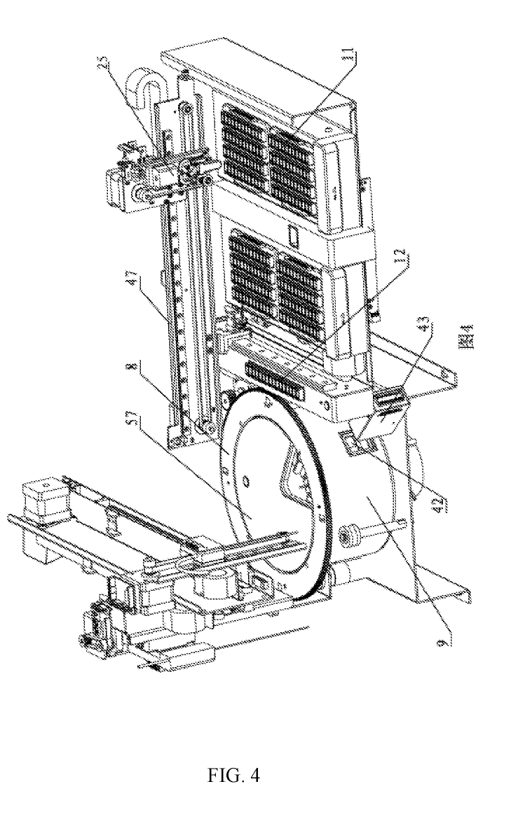

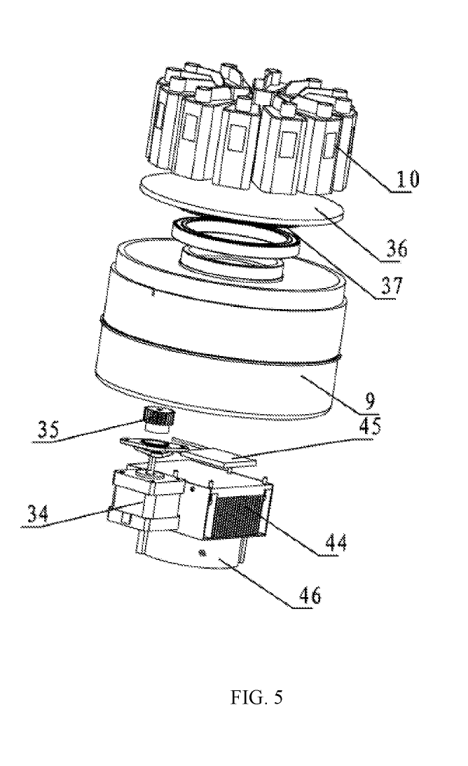

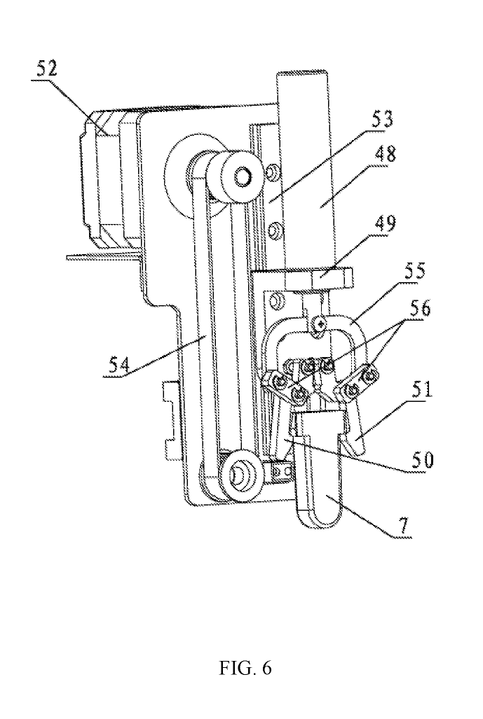

By referring to FIG. 1, FIG. 2, FIG. 3, FIG. 4, FIG. 5 and FIG. 6, a full-automatic instant check meter, comprising a shell (not shown in the figure), a frame body 1, a sample rack 2, a sample input device, a shaking device, a detection card stack, a sample reaction device, a detection card conveying device, a detection device and a control panel (not shown in the figure), wherein the sample input device, the shaking device, the detection card stack, the sample reaction device, the detection card conveying device and the detection device are all arranged on the frame body; the sample rack is arranged in the sample input device, and a sample test tube 3 is placed on the sample rack 2; the shaking device is arranged behind the sample input device; and the sample reaction device, the detection card conveying device and the detection device are all arranged behind the shaking device. The sample input device comprises a push rod mechanism, a limiting mechanism and an induction mechanism; the shaking device comprises a first barcode printer 4, a rotating mechanism, a vacuum blood vessel shaking mechanism and a peripheral blood vessel shaking mechanism; and the rotating mechanism, vacuum blood vessel shaking mechanism and peripheral blood vessel shaking mechanism are all fixed on the frame body; the peripheral blood vessel shaking mechanism is arranged below the vacuum blood vessel shaking mechanism; the rotating mechanism is arranged on the side of vacuum blood vessel shaking mechanism; the detection card stack comprises a detection card 5, a detection card stack 6 and a scanning mechanism; the detection card 5 is arranged in the detection card stack 6, and the scanning mechanism is arranged behind the detection card stack 6; the sample reaction device comprises a reaction cup 7, a flowing water ring 8, a reagent bin 9, a reagent bottle 10, a sampling arm mechanism, a gripping mechanism, a reaction cup stack area 11 and a metal bath area 12; the reagent bottle 10 is arranged in the reagent bin 9; the flowing water ring 8 is arranged on the upper end face of the reagent bin 9, and the reaction cup 7 is arranged above the flowing water ring 8; the sampling arm mechanism is arranged above the reagent bin 9 and fixed on the frame body 1. The gripping mechanism is arranged above the reaction cup stack area 11 and metal bath area 12 and fixed on the frame body 1; the detection card conveying device comprises a detection card conveying arm, a buffer cartridge 13 and a card extraction mechanism; the detection card conveying arm is arranged behind the detection card stack 6 and fixed on the frame body 1; the card extraction mechanism is arranged on the detection card conveying arm in a sliding manner; the detection device comprises a detection mechanism and a detection card throwing mechanism; the detection mechanism is arranged above the detection card throwing mechanism; the buffer cartridge is arranged between the detection mechanism and the card extraction mechanism; the control panel is fixed to the frame body 1; the push rod mechanism, limiting mechanism, induction mechanism, first barcode printer, rotating mechanism, vacuum blood vessel shaking mechanism, peripheral blood vessel shaking mechanism, scanning mechanism, sampling arm mechanism, gripping mechanism, metal bath area, detection card conveying arm, buffer cartridge, card extraction mechanism, detection mechanism and detection card throwing mechanism are electrically connected on the control panel respectively.

In this embodiment, the push rod mechanism comprises a first push rod 14, a second push rod 15, a third push rod 16 and a fourth push rod 17, respectively driven by the first motor, second motor, third motor and fourth motor; the first push rod 14 and the third push rod 16 are used to move the sample rack 2 longitudinally, and the second push rod 15 and the fourth push rod 17 are used to move the sample rack 2 transversely; the limiting mechanism comprises the first limit switch 18 that limits the sample rack longitudinally and the second limit switch 19 that limits the sample rack transversely; the sample input device further comprises a limiting door 20 for butting and a beam photoelectric sensor 21 is arranged above the limiting door 20.

In this embodiment, the rotating mechanism comprises a first DC motor 22 and a rotating friction head 23; the first DC motor 22 is connected with the frame body 1 in sliding manner at the vertical direction; the vacuum blood vessel shaking mechanism comprises a guide block 24 and a gripper 25, the guide block 24 is fixed on the frame body 1 and the gripper 25 is connected with the frame body 1 in sliding manner at the vertical direction; a check block 26 is provided on the gripper 25 and the check block 26 rotates to drive the gripper 25 to rotate under the guiding of the guide block 24; the peripheral blood vessel shaking mechanism comprises an installation platform 27, a second DC motor 28, a lead screw 29, a fixed block 30, a brushless motor 31 and a fixing piece 32; the installation platform 27 is fixed on the frame body 1, the fixed block 30 is connected with the installation platform 27 in a sliding manner driven by the second DC motor 28 and the lead screw; an eccentric block 33 is arranged on the brushless motor 31 to drive the fixing piece 32 to vibrate when the brushless motor 31 rotates.

In this embodiment, the reagent bin 9 is arranged on the frame body 1; the reagent bin 9 is internally provided with a fifth motor 34 and a first gear 35 is arranged at the output end of the fifth motor 34. A reagent tray 36 is arranged in the reagent bin 9 and a ring gear 37 is arranged on the lower surface of the reagent tray 36. The first gear 35 is meshed with the ring gear 37; the reagent bottle 10 is arranged on the reagent tray 36; the flowing water ring 8 is evenly distributed with four reaction cup jacks 38; the reaction cup 7 is inserted into the reaction cup jacks 38; the flowing water ring 8 is provided with card latch 39 and a sixth motor 40 outside. The output upper end of the sixth motor 40 is provided with a second gear 41 which is meshed with the flowing water ring 8; the metal bath area 12 is located at the side of the reagent bin 9, the reaction cup stack area 11 is located at the side of the metal bath area 12; an opening 42 is arranged on the reagent bin 9; the second barcode printer 43 is arranged at the opening 42 and fixed on the frame body 1; a radiator 44 is arranged on the side of the fifth motor 34 inside the reagent bin 9; chilling chips 45 are arranged above the radiator 44 and fans 46 are arranged below the radiator 44.

In this embodiment, the gripping mechanism comprises a gripper arm 47, an electromagnet 48, a sliding block 49, a left grab block 50 and a right grab block 51; a seventh motor 52 and a sliding rail 53 are arranged on the gripper arm 47, the sliding block 49 is connected with the sliding rail 53 in a sliding manner and connected the seventh motor 52 via a synchronous belt 54; the electromagnet 48 is arranged on the sliding block 49 and a first connecting rod 55 is arranged at the lower end of the electromagnet 48; the left grab block 50 and right grab block 51 are pivotally connected with the first connecting rod 55 via the second connecting rod 56 respectively.

In this embodiment, a reagent bin cover 57 is arranged above the reagent bottle 10 and the reagent bin cover 57 is provided with a sector-shaped notch 58.

In this embodiment, the detection module comprises an immune colloidal gold detection module (not shown in the figure) and an immunofluorescence detection module (not shown in the figure), and the immune colloidal gold detection module and immunofluorescence detection module are both arranged above the detection card 5.

Of course, the detection mechanism may also comprise one or two immune colloidal gold detection modules, or comprises one or two immunofluorescence detection modules, or comprises one or two IMB detection modules, or comprises one or two immunofluorescence particle modules, or comprises one immune colloidal gold detection module, one immunofluorescence detection module and one IMB detection module, or comprises one immune colloidal gold detection module, one immunofluorescence detection module, one IMB detection module and one immunofluorescence particle module, all of which are equivalent and fall within the scope of protection in the present invention.

1. A fully-automatic instant check meter, comprising a shell, a frame body, a sample rack, a sample input device, a shaking device, a detection card stack, a sample reaction device, a detection card conveying device, a detection device and a control panel, wherein the sample input device, the shaking device, the detection card stack, the sample reaction device, the detection card conveying device and the detection device are all arranged on the frame body; the sample rack is arranged in the sample input device, and a sample test tube is placed on the sample rack; the shaking device is arranged behind the sample input device; and the sample reaction device, the detection card conveying device and the detection device are all arranged behind the shaking device, the sample input device comprises a push rod mechanism, a limiting mechanism and an induction mechanism; the shaking device comprises a first barcode printer, a rotating mechanism, a vacuum blood vessel shaking mechanism and a peripheral blood vessel shaking mechanism; and the rotating mechanism, vacuum blood vessel shaking mechanism and peripheral blood vessel shaking mechanism are all fixed on the frame body; the peripheral blood vessel shaking mechanism is arranged below the vacuum blood vessel shaking mechanism; the rotating mechanism is arranged on the side of vacuum blood vessel shaking mechanism; the detection card stack comprises a detection card, a detection card stack and a scanning mechanism; the detection card is arranged in the detection card stack, and the scanning mechanism is arranged behind the detection card stack; the sample reaction device comprises a reaction cup, a flowing water ring, a reagent bin, a reagent bottle, a sampling arm mechanism, a gripping mechanism, a reaction cup stack area and a metal bath area; the reagent bottle is arranged in the reagent bin; the flowing water ring is arranged on the upper end face of the reagent bin, and the reaction cup is arranged above the flowing water ring; the sampling arm mechanism is arranged above the reagent bin and fixed on the frame body, the gripping mechanism is arranged above the reaction cup stack area and metal bath area and fixed on the frame body; the detection card conveying device comprises a detection card conveying arm, a buffer cartridge and a card extraction mechanism; the detection card conveying arm is arranged behind the detection card stack and fixed on the frame body; the card extraction mechanism is arranged on the detection card conveying arm in a sliding manner; the detection device comprises a detection mechanism and a detection card throwing mechanism; the detection mechanism is arranged above the detection card throwing mechanism; the buffer cartridge is arranged between the detection mechanism and the card extraction mechanism; the control panel is fixed to the frame body; the push rod mechanism, limiting mechanism, induction mechanism, first barcode printer, rotating mechanism, vacuum blood vessel shaking mechanism, peripheral blood vessel shaking mechanism, scanning mechanism, sampling arm mechanism, gripping mechanism, metal bath area, detection card conveying arm, buffer cartridge, card extraction mechanism, detection mechanism and detection card throwing mechanism are electrically connected on the control panel respectively.

2. The fully-automatic instant check meter according to claim 1, wherein the push rod mechanism comprises a first push rod, a second push rod, a third push rod and a fourth push rod, respectively driven by the first motor, second motor, third motor and fourth motor; the first push rod and the third push rod are used to move the sample rack longitudinally, and the second push rod and the fourth push rod are used to move the sample rack transversely; the limiting mechanism comprises the first limit switch that limits the sample rack longitudinally and the second limit switch that limits the sample rack transversely; the sample input device farther comprises a limiting door for butting and a beam photoelectric sensor is arranged above the limiting door.

3. The fully-automatic instant check meter according to claim 2, wherein the rotating mechanism comprises a first DC motor and a rotating friction head; the first DC motor is connected with the frame body in sliding manner at the vertical direction; the vacuum blood vessel shaking mechanism comprises a guide block and a gripper, the guide block is fixed on the frame body and the gripper is connected with the frame body in sliding manner at the vertical direction; a check block is provided on the gripper and the check block rotates to drive the gripper to rotate under the guiding of the guide block; the peripheral blood vessel shaking mechanism comprises an installation platform, a second DC motor, a lead screw, a fixed block, a brushless motor and a fixing piece; the installation platform is fixed on the frame body, the fixed block is connected with the installation platform in a sliding manner driven by the second DC motor and the lead screw; an eccentric block is arranged on the brushless motor to drive the fixing piece to vibrate when the brushless motor rotates.

4. The fully-automatic instant check meter according to claim 3, wherein the reagent bin is arranged on the frame body; the reagent bin is internally provided with a fifth motor and a first gear is arranged at the output end of the fifth motor, a reagent tray is arranged in the reagent bin and a ring gear is arranged on the lower surface of the reagent tray, the first gear is meshed with the ring gear; the reagent bottle is arranged on the reagent tray; the flowing water ring is evenly distributed with four reaction cup jacks; the reaction cup is inserted into the reaction cup jacks; the flowing water ring is provided with card latch and a sixth motor outside, the output upper end of the sixth motor is provided with a second gear which is meshed with the flowing water ring; the metal bath area is located at the side of the reagent bin, the reaction cup stack area is located at the side of the metal bath area; an opening is arranged on the reagent bin; the second barcode printer is arranged at the opening and fixed on the frame body; a radiator is arranged on the side of the fifth motor inside the reagent bin; chilling chips are arranged above the radiator and fans are arranged below the radiator.

5. The fully-automatic instant check meter according to claim 4, wherein the gripping mechanism comprises a gripper arm, an electromagnet, a sliding block, a left grab block and a right grab block; a seventh motor and a sliding rail are arranged on the gripper arm, the sliding block is connected with the sliding rail in a sliding manner and connected the seventh motor via a synchronous belt; the electromagnet is arranged on the sliding block and a first connecting rod is arranged at the lower end of the electromagnet; the left grab block and right grab block are pivotally connected with the first connecting rod via the second connecting rod respectively.

6. The fully-automatic instant check meter according to claim 5, wherein a reagent bin cover is arranged above the reagent bottle and the reagent bin cover is provided with a sector-shaped notch.

7. The fully-automatic instant check meter according to claim 6, wherein the detection module comprises one or more of an immune colloidal gold detection module, an immunofluorescence detection module, an IMB detection module, and an immunofluorescence particle module; the immune colloidal gold detection module or immunofluorescence detection module or IMB detection module or immunofluorescence particle module is arranged above the detection card bracket.
